(19) United States
(12) Patent Application Publication
(10) Pub. No.: US 2008/0227190 A1
(43) Pub. Date: Sep. 18, 2008

(54) Cell Expansion Apparatus with Plate Bioreactor

(75) Inventor: Glen Delbert ANTWILER, Lakewood, CO (US)

Correspondence Address: GAMBRO BCT, INC;IP DEPARTMENT, 10810 WEST COLLINS AVE, LAKEWOOD, CO, 80215, US

(73) Assignee: GAMBRO BCT, INC., Lakewood, CO (US)

(21) Appl. No.: 12/047,451

(22) Filed: Mar. 13, 2008

Related U.S. Application Data
(60) Provisional application No. 60/894,791, filed on Mar. 14, 2007.

(51) Int. Cl. C12M 1/12

(52) U.S. Cl. 435/297.5

(57) ABSTRACT

A disposable apparatus for cell expansion, having at least one bioreactor. The bioreactor has a cellular growth area and a supply area, the cellular growth area being adjacent a plate and separated from said supply area by a membrane. The bioreactor comprises a plurality of plates. The plates have a roughened surface or a corrugated surface. At least a sheet of membrane separates adjacent plates. Two sheets between adjacent plates may be separated by a mesh.

The present invention is directed toward an apparatus for cell expansion including a stacked plate bioreactor. The plates of the bioreactor may be flat with corrugated surfaces. One or more membranes between the plates separate cell expansion areas from areas containing fluids for chemical exchange. Cell expansion areas may be adjacent the stacked plates or between membranes.

### BACKGROUND OF THE INVENTION

Stem cells can be expanded from a few donor cells in a cell expansion apparatus. The resulting multiplied cells can be used to repair or replace damaged or defective tissues. Stem cells have broad clinical applications for a wide range of diseases. Recent advances in the area of regenerative medicine have demonstrated that stem cells have unique properties such as high proliferation rates and self-renewal capacity, ability to maintain an unspecialized cellular state, and the ability to differentiate into specialized cells under particular conditions.

As an important component of regenerative medicine, bioreactor systems play an important role in providing optimized environments for cell expansion. The bioreactor provides for efficient nutrient supply to the cells and for removal of metabolites, as well as furnishing a pysiochemical environment conducive to cell growth. In particular, foreign cells, such as air-borne pathogens, must be excluded from the cell-growth areas of the bioreactor. A bioreactor should be provided with relatively large surface areas available for cell adhesion during the cell expansion process. It must be possible, however, to harvest the expanded cells without damage.

### SUMMARY OF THE INVENTION

The present invention comprises a disposable apparatus for cell expansion, having at least one bioreactor. The bioreactor has a cellular growth area and a supply area, the cellular growth area being separated from said supply area by a membrane. The membrane inhibits migration of cells from the cellular growth area to the supply area and permits migration of certain chemical compounds from the cellular growth area to the supply area and of certain other chemical compounds from the supply area to the cellular growth area. At least one oxygenator is in fluid communication with the supply area, and a plurality of bags is in fluid communication with the cellular growth area, the bags providing fluids to the cellular growth area. A fluid recirculation path is in fluid communication with the cellular growth area.

The bioreactor comprises a plurality of plates stacked to form a reactor. One or more membranes are sandwiched between adjacent plates. The membranes separate supply areas from cellular growth areas. Cellular growth areas may be provided adjacent a plate and at least one membrane or between two membranes. The plates may have a corrugated surface providing increased surface area for cell adhesion.

Another aspect of the invention comprises a method of expanding cellular matter, the method comprising providing at least one bioreactor, the bioreactor having a cellular growth area and a supply area, and the cellular growth area being separated from the supply area by a membrane, the membrane being adapted to inhibit migration of cells from said cellular growth area to said supply area and to permit migration of certain chemical compounds from said cellular growth area to said supply area and of certain other chemical compounds from said supply area to said cellular growth area. The bioreactor comprises a plurality of plates stacked to form a reactor. Membranes are sandwiched between adjacent plates. The method further comprises conducting a fluid containing cellular matter into the cellular growth area, providing oxygenated fluid to the supply area to maintain conditions conducive for cell growth in the cellular growth area, and flushing expanded cellular matter from the cellular growth area of the bioreactor.

These and other features and advantages of the present invention will be apparent from following detailed description, taken with reference to the attached drawings and claims.

### BRIEF DESCRIPTION OF THE DRAWINGS

FIG. 1 is a schematic description of a cell expansion apparatus.

FIG. 2 is a perspective view of a stacked plate bioreactor.

FIG. 3 is a plan view of a plate for the bioreactor of FIG. 2.

FIG. 4 is a cross sectional view of the plate of FIG. 3, taken along line 4-4.

FIG. 5 is a cross sectional view of the plate of FIG. 3, taken along line 5-5.

FIG. 6a is an exploded side view of two plates with two intervening membranes.

FIG. 6b is an exploded side view of two plates with one intervening membrane.

FIG. 7 is a plan view of a spacer, showing a first side.

FIG. 8 is a plan view of the spacer of FIG. 7, showing a second side.

FIG. 9 is a cross-sectional view of the spacer of FIG. 7, taken along line 9-9 of FIG. 7.

FIG. 10 is a cross-sectional view of the spacer of FIG. 7, taken along line 10-10 of FIG. 8.

### DETAILED DESCRIPTION OF THE INVENTION

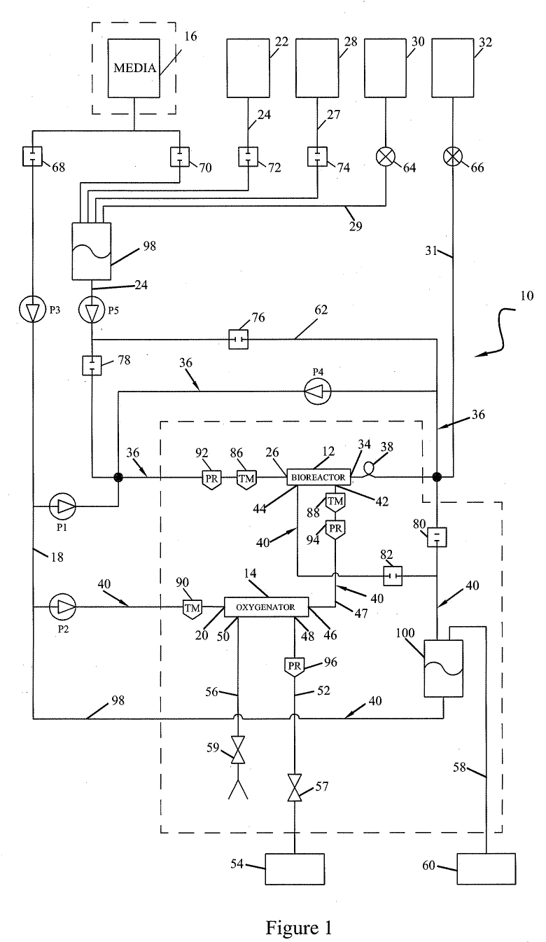

In the following description of the invention and in the accompanying drawings, like numerals refer to like parts. FIG. 1 shows a schematic diagram of a cell expansion apparatus 10. The cell expansion apparatus comprises a bioreactor 12 wherein selected cells multiply and an oxygenator 14 wherein supply fluid is provided with oxygen.

### Bioreactor Module

A bioreactor 12 may be made of flat sheet membranes sandwiched between plates and enclosed in a housing. The plates are preferably flat and have corrugated surfaces. The sheets may be made of a biocompatible polymeric material such as a blend of polyamide, polyarylethersulfone and polyvinylpyrrolidone. Depending upon the type of cells to be expanded in the bioreactor, the sheets may or may not be treated with a substance to enhance cell growth and/or adherence to the membrane. The bioreactor housing has at least four openings into the interior of the housing. Two open into the cell expansion areas, and two open into the supply space, adjacent the cell expansion areas.

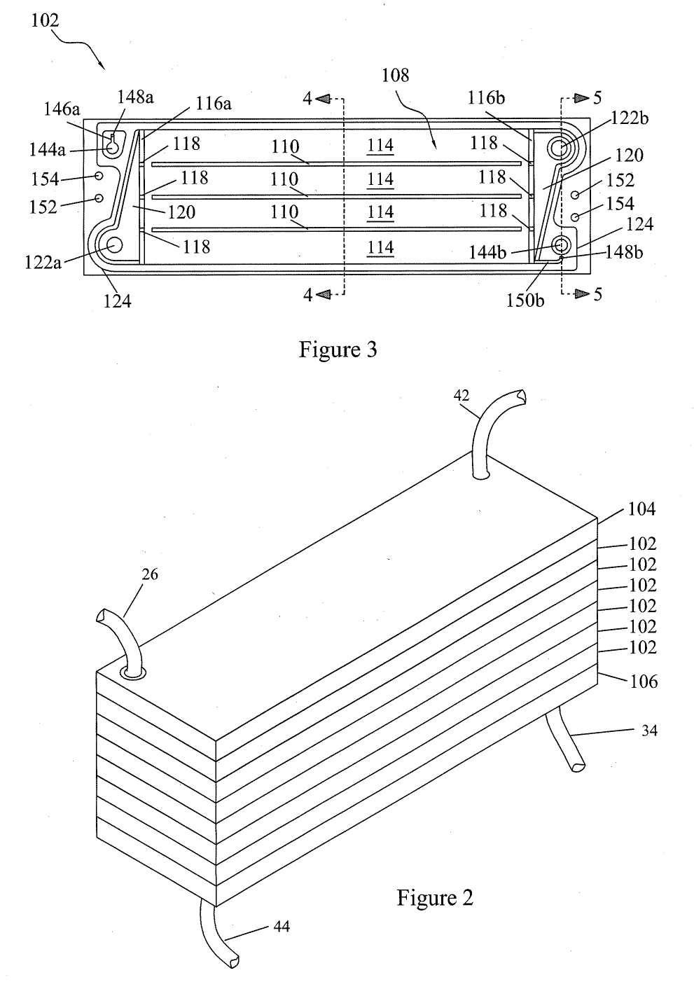

FIG. 2 shows a flat sheet bioreactor 12 according to the present invention. The bioreactor 12 comprises a stack of symmetrical plates 102. A top plate 104 and a bottom plate 106 may have similar two-sided configurations like the symmetrical plates 102 or preferably may be essentially flat and featureless on their exposed top and bottom sides, as shown. A supply inlet port 42 enters the bioreactor at an upper corner and a supply outlet port 44 leaves the bioreactor at an opposite lower corner. As will be explained below, supply fluids pass from the supply inlet port 42 to the supply outlet port 44 and provide both nutrients and oxygen and also remove waste materials. Nutrients and oxygen pass into a cell expansion area across a membrane, just as waste materials pass out of the cell expansion area across the membrane. The cell expansion area is provided with cell expansion media, including living cells, through the cell expansion inlet port 26. Fluids of the cell expansion media, including some cells, leave the cell expansion area through the cell expansion outlet port 34.

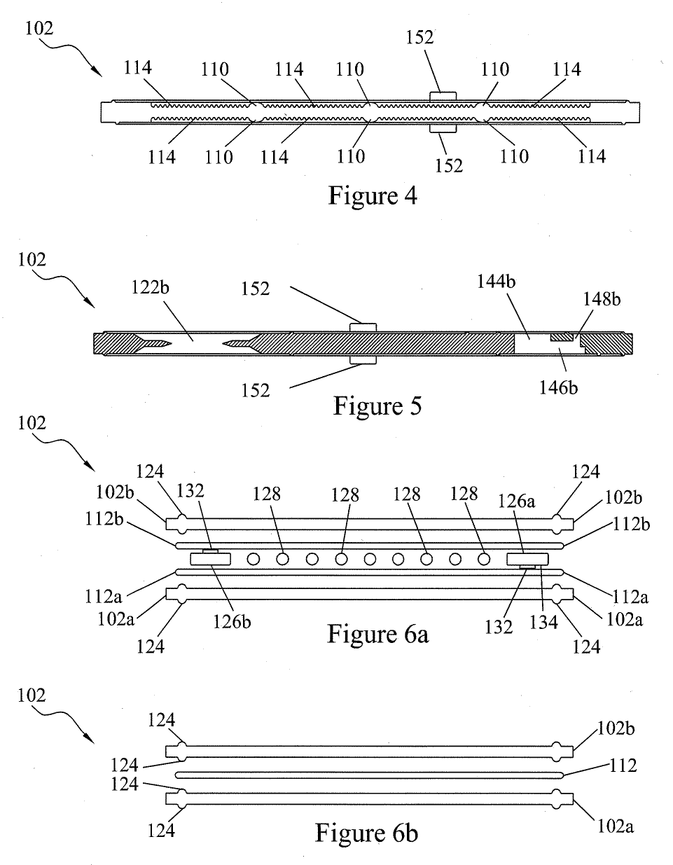

An exemplary plate 102 is shown in FIGS. 3, 4 and 5. The plan view of FIG. 3 serves as both a top and a bottom view, as the plate is symmetrical around the through-section line 4-4. In other words, rotating the plate 102 by 180 degrees around line 4-4 gives the same configuration as presently shown in FIG. 3. Each plate 102 comprises a rectangular central field 108 having a plurality of longitudinal ribs 110. The ribs 110 provide support for a membrane 112 (see FIG. 6b) or membranes 112a, 112b (see FIG. 6a), more fully described below, and separate corrugated areas 114 on the central field 108. Corrugations run parallel to the ribs 110 such that fluid flows along the corrugations from one end of the central field to the other. The corrugations provide increased surface area to which cells may attach during the cell expansion or cell growth process and further prevent adjacent membranes from sealing to the plate. Although corrugations are the preferred configuration, other surface features may be used. A roughened surface might be provided by corrugations, by dimples, by ridges, or by cross-hatched grooves, for example, or by other surface features increase the surface area and decrease contact area presented to adjacent membrane. At each end of the central field 108, there is a slot 116a, 116b (see FIG. 3) that allows fluid to flow from one side of a plate to another. Struts 118 may provide additional stability. Adjacent each slot 116a, 116b is a triangular trough 120 that allows fluid to flow from a through bore 122a on one end of the plate 102 to another through bore 122b on the other end of the plate. At least one membrane 112 is placed on the plate. A circumferential bead 124 FIG. 3 and also FIGS. 6a and 6b) on the plate will seal the membrane or membranes between two adjacent plates.

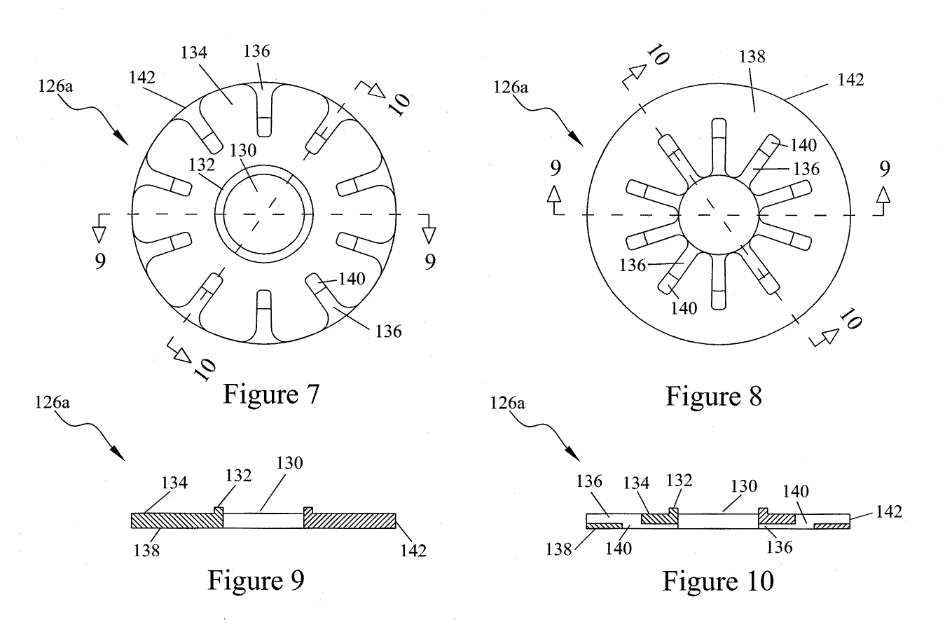

In the preferred embodiment, two membranes 112a and 112b, as shown in FIG. 6a, are inserted between adjacent plates 102a and 102b. The membranes are chosen with porosity and hydrolytic properties to allow nutrients and metabolites such as glucose, lactate, oxygen, carbon dioxide to pass from one side of a membrane to another, but preferably to limit passage of high-cost growth factors, which are retained in cell expansion areas. The membrane may also be chosen to provide cell attachment properties, thus providing additional surface area for cell growth. Spacers 126a and 126b provide a flow path for supply fluid from the inlet through bore 122a into a space between the two membranes and out the outlet through bore 122b. The space between the membranes contains a polypropylene mesh 128 or similar structure to hold the membranes apart while allowing flow between the membranes. The configuration of a suitable spacer 126a is shown in FIGS. 7, 8, 9 and 10. The spacer 126a has a central hole 130 with a narrow circumferential lip 132 on a first side 134 of the spacer. A plurality of radial channels 136 extend from the hole 130 adjacent a second side 138 (FIG. 8) of the spacer, open to both sides of the spacer in a central area 140, and extend to an outer edge 142 of the spacer adjacent the first side 134 of the spacer. FIG. 6a is an exploded end view of two plates 102a, 102b with membranes 112a, 112b and spacers 126a, 126b. As shown in FIG. 6a, the first spacer 126a is placed on the first membrane 112a with the first side 134 and lip 132 facing down toward the inlet bore 122a and is sealed to the membrane with adhesive. A hole is made in the first membrane 122a corresponding to the hole in the spacer and the inlet bore 122a. Supply fluid will be able to flow through the inlet bore and the hole, out the radial channels 136, and into the space between the membranes. At the other end of the plate, a second spacer 126b is placed over the outlet bore 122b with the first side 134 and rim 132 facing up towards the upper plate 102b. A second membrane 112b is placed over the mesh 128 and first membrane 112a. Another hole is made in the second membrane 122b corresponding to the hole in the second spacer 126b and the outlet bore 122b in the upper plate 102b. Supply fluid will be able to flow out of the space between the membranes, into the radial channels 136, through the hole and the outlet bore and into the next set of plates. This pattern may be repeated as often as desired to create the stacked bioreactor.

A separate fluid path is provided for the cell media outside the two membranes and adjacent the corrugated surfaces 114. Cell media enters the bioreactor through the cell expansion inlet port 26 and fills a column comprising a series of holes 144a (FIG. 3) in successive plates 102. Cell media will flow through a transverse spur 146a seen in the upper left area of FIG. 3, then through a vertical pipe 148a, and into a J-shaped passageway (not shown, but see passageway 150b) on the opposite side of the plate 102. The J-shaped passageway can be seen in the lower right area of FIG. 3, where the passageway 150b connects to a vertical pipe 148b and a transverse spur 146b, as also shown in FIG. 5. When two plates are stacked together, adjacent J-shaped passageways and transverse spurs join to form common fluid flow paths. Cell media flows from the inlet column 144a through a plurality of such paths into the slots 116a, then between a membrane and an adjacent corrugated surface to slots 116b, where the fluid is collected through a plurality of paths comprising J-shaped passageways 150b, vertical pipes 148b and transverse spurs 146b into an outlet column 144b. Cell media leaves the bioreactor through the outlet port 34.

The plates 102 are stacked together and are aligned by tabs 152 that fit into holes 154. The tabs and holes are slightly asymmetrical with respect to a centerline of the plate 102, so that when plates are stacked together, only sides with congruent features may be placed next to each other.

Cells may be grown in the cell expansion areas. Minimizing the volume of the cell expansion areas reduces the quantity of expensive media and expensive cytokines/growth factors required. The semi-permeable membrane allows transfer of metabolic components, waste and gases between the supply and cell expansion areas. The molecular transfer characteristics of the membranes are chosen to minimize loss of expensive reagents from the cell expansion side, while allowing metabolic waste products to diffuse through the membrane into the supply side to be removed. The supply space between the membranes carries nutrients to the cells in the cell expansion space, removes waste byproducts and maintains gas balance. The bioreactor may be attached to the rest of the disposable set with connectors made of polyurethane.

In another embodiment, a single membrane 112 may be placed between adjacent plates 102a, 102b, as shown in FIG. 6b. Fluid on one side of the membrane would be supply media, while fluid on the opposite side of the membrane would be cell media.

### Oxygenator

The oxygenator 14 used may be any commercially available oxygenator. One alternative oxygenator that may be used is a hollow fiber Oxy-Cell Mate oxygenator having a fiber count of 1820, an internal fiber diameter of 280 μm, an outer fiber diameter of 386 μm and an intercapillary fluid volume of 16 mL. The hollow fibers of the oxygenator are enclosed in a housing having four port openings. An oxygenator supply inlet port 20 and an oxygenator supply outlet port 46 are fluidly connected to the interior of the hollow fibers. A second inlet port 48 and a second outlet port 50 are fluidly connected to the space surrounding the hollow fibers (the “supply space”). The supply inlet port 20 of the oxygenator 14 connects the supply inlet line 18 to the oxygenator 14 to deliver either fresh media from the supply media bag 16 or recirculated supply media from the bioreactor to the oxygenator 14. A supply line 47, connected to the oxygenator outlet port 46, delivers oxygenated supply media to the supply inlet port 42 on the bioreactor 12.

A gas line 52, connected to the oxygenator gas inlet port 48, is coupled to a source of gas 54, such as oxygen or another appropriate gas or mixture of gases. The oxygenator gas outlet port 50 is open to the atmosphere through an exhaust line 56. Both the gas line 52 and the exhaust line 56 have 0.22μ in-line filters 57, 59 to prevent microbes from entering and contaminating the closed system.

### Supply Media Bag and Supply Media Inlet Line

A supply media bag 16, which contains fluid for the supply side of the bioreactor, may be connected via a portion of the supply inlet line 18 to the supply inlet port 20 of the oxygenator 14. The supply inlet line 18 brings fresh supply media to the oxygenator 14 to be oxygenated. The supply inlet line 18 may be made of polyvinyl chloride with fluorinated ethylene propylene (PVC/FEP).

### Cell Expansion Media Bag and Cell Expansion Media Inlet Line

A cell expansion media bag 22, which contains fluid for the cell expansion side of the bioreactor, may be connected via a cell expansion inlet line 24 to the cell expansion inlet port 26 of the bioreactor 12. The cell expansion inlet line 24 brings fresh cell expansion media to the cell expansion side of the bioreactor. The cell expansion inlet line 24 may also be made of PVC/FEP.

### Vent Bag

A vent bag 28 may be connected to the disposable set via flexible tubing 27 to collect any air initially in the system before the system is filled with media and biologic fluids, including cells.

### Cell Input Bag

A cell input bag 30 contains cells to be added to the bioreactor 12. The cell input bag 30 is connected to the cell expansion inlet line 24, which delivers cells into the cell expansion areas, via a cell input line 29.

### Cell Harvest Bag

When the cells are ready to be harvested, they are flushed out of the cell expansion outlet port 34 of bioreactor 12 through cell harvest line 31 and into a cell harvest bag 32.

### Cell Expansion Recirculation/Reseeding Tubing Loop

The disposable tubing set also may include a length of tubing which acts as a cell expansion circulation loop 36. The cell expansion media flows out of the bioreactor 12 from the cell expansion outlet port 34 through tubing loop 36 and back into the bioreactor through the cell expansion inlet port 26. This loop 36 is used to recirculate the cell expansion media though the cell expansion areas. It may also be used to flush the cells out of the cell expansion areas and reseed/redistribute them throughout the cell expansion areas for further growth.

The cell expansion recirculation loop 36 may contain sample tubing 38, for example, an additional length of tubing. This additional tubing enables small pieces of the tubing to be sterilely removed from the disposable set and the media inside to be tested for cell concentration as well as for markers of cellular metabolism such as pH, glucose, lactate, electrolytes, oxygen and carbon dioxide content.

### Supply Recirculation Loop

A supply recirculation loop 40 allows the media on the supply side of the bioreactor to be recirculated. The supply recirculation loop 40 allows supply media to flow out of the bioreactor from the supply outlet port 44, through the oxygenator inlet port 20, out the oxygenator outlet port 46, and back into the bioreactor through the supply inlet port 42. This loop may be used to recirculate the supply media that surrounds the cell expansion areas.

### Waste Bag

Both cell expansion media and supply media containing metabolic breakdown products from cell growth are removed from the system via tubing 58 into a waste bag 60.

### Pump Loops

As shown in FIG. 1, the tubing set may engage three or more pump loops that correspond to the location of peristaltic pumps on the cell expansion apparatus. In one embodiment, the tubing set 10 may have five pump loops, corresponding to pumps P1-P5 on the apparatus.

### Cassette

A cassette for organizing the tubing lines and which may also contain tubing loops for the peristaltic pumps may also be included as part of the disposable. In order to control the passage of fluid through the disposable 10, manually operated clamps 64, 66 may be provided. In addition, microprocessor-controlled pinch valves 68, 70, 72, 74, 76, 78, 80, and 82 may be coupled to selected tubes of the disposable. Temperature sensors 86, 88, 90 and pressure sensors 92, 94, and 96 can be connected to selected tubes of the disposable 10 and placed in electrical communication with a microprocessor (not shown). It is to be understood that pumps, temperature sensors, pressure sensors and pinch valves are preferably connected to the disposable set only temporarily by contact. Manual clamps, on the other hand, are usually mounted on their respective tubes and may be delivered with the disposable.

With the disposable apparatus 10 mounted in an incubator, fluid supply media fills supply portions of the disposable apparatus 10, including connecting tubes, first and second drip chambers 98, 100, the oxygenator 14 and the bioreactor 12. Pumps P1, P2, P3 and P4 may be selectively activated to force fluid into sections of the disposable apparatus to prime the apparatus. After priming, intercellular media and cells, for example mesenchymal stem cells, may be added from bags 22, 30 through the first drip chamber 98 and conducted into a cell expansion area of the bioreactor 12 and related tubing, including recirculation path 36 and sample means 38. The hermetically sealed condition of the disposable apparatus 10 is maintained by providing a vent bag 28 coupled to the first drip chamber 98 to accommodate variations in flow from the supply media bag 16, the cell expansion media bag 22 and the cell input bag 30.

Driven by pump P2, supply fluid passes through the oxygenator 14 where the fluid is infused with gas. The fluid then passes into the supply area of the bioreactor 12. The supply area is separated from the cell expansion area by at least one membrane that allows oxygen and other desirable chemical components to pass into the cell expansion area and allows waste products of the cell expansion process to pass by osmosis out of the cell expansion area. The cellular matter does not cross the membrane. Temperature sensor 88 and pressure sensor 94 monitor the status of the fluid flowing through the supply area of the bioreactor. Temperature sensor 90 monitors the temperature of the fluid entering the oxygenator 14. An appropriate gas, such as oxygen or a gas mixture, is conducted through the oxygenator 14 at a pressure monitored by pressure sensor 96. The gas is preferably medical grade and is also isolated from ambient air by 0.22 micron filters 57, 59.

The pumps are preferably peristaltic pumps. In addition to the manual clamps and automatically controlled valves, the pumps also act as valves, preventing flow of fluid past the pump when the pump is not actively driven. Therefore, when pump P1 is not in operation and valves 76, 78 are closed, the recirculation loop 36 is formed through the bioreactor 12 and pump P4. Conditions in this recirculation loop, where cells may be growing or through which cells may be flushed, are monitored with temperature sensor 86 and pressure sensor 92 and by taking fluid samples through a sample port (not shown).

The sample loop 38 allows discrete samples of cell-containing fluid to be removed from the cell expansion area from time to time. The samples may then be tested to determine the state of cell growth in the cell expansion area. When sufficient cell replication has taken place, as determined by analysis of the sample, the contents of the bioreactor can be harvested into the cell harvest bag 32. The expanded cellular material would then be available for therapeutic and other purposes.

The foregoing description of the present invention has been presented for purposes of illustration and description. Furthermore, the description is not intended to limit the invention to the form disclosed herein. Consequently, variations and modifications commensurate with the above teachings, and skill and knowledge of the relevant art, are within the scope of the present invention. The embodiments described hereinabove are further intended to explain best modes known of practicing the invention and to enable others skilled in the art to utilize the invention in such, or other embodiments and with various modifications required by the particular application(s) or use(s) of the present invention. It is intended that the appended claims be construed to include alternative embodiments to the extent permitted by the prior art.

I claim

1. A disposable apparatus for cell expansion, said apparatus comprising
at least one bioreactor, said bioreactor having a cellular growth area and a supply area, said cellular growth area being separated from said supply area by a membrane, said membrane being adapted to inhibit migration of cells from said cellular growth area to said supply area and to permit migration of certain chemical compounds from said cellular growth area to said supply area and of certain other chemical compounds from said supply area to said cellular growth area, wherein
said bioreactor comprises a plurality of plates, said plates having a roughened surface adjacent a cellular growth area and
said membrane comprises a sheet, at least one sheet being between two adjacent plates;
at least one oxygenator in fluid communication with said supply area;
a plurality of bags in fluid communication with said cellular growth area, said bags being adapted to provide fluids to said cellular growth area;
a fluid recirculation path in fluid communication with said cellular growth area.

2. The disposable apparatus of claim 1 further comprising at least two sheets of membrane between two adjacent plates, wherein said supply area is between said two sheets of membrane.

3. The disposable apparatus of claim 2 further comprising a support structure between said at least two sheets of membrane.

4. The disposable apparatus of claim 3 wherein said support structure is a mesh.

5. The disposable apparatus of claim 2 further comprising means for directing fluid into an area between adjacent sheets of membrane and means for directing another fluid into an area between a sheet of membrane and a plate.

6. The disposable apparatus of claim 5 wherein the fluid between the membrane and a plate contains cells and the fluid between the two membranes supplies nutrients.

7. The disposable of claim 1 wherein at least a surface of said plate facing a sheet of membrane has a corrugated surface.

8. The disposable of claim 7 wherein at least a surface of said plate facing a sheet of membrane has a plurality of grooves, whereby the surface area of said plate is increased.

9. The disposable of claim 8 wherein said grooves are parallel to each other.

10. A cell expansion system comprising
at least one bioreactor, said bioreactor having a cellular growth area and a supply area, said cellular growth area being separated from said supply area by a membrane, said membrane being adapted to inhibit migration of cells from said cellular growth area to said supply area and to permit migration of certain chemical compounds from said cellular growth area to said supply area and of certain other chemical compounds from said supply area to said cellular growth area, wherein
said bioreactor comprises a plurality of plates, each plate having a roughened surface adjacent a cellular growth area, and
said membrane comprises a sheet, at least one sheet being between two adjacent plates;
at least one oxygenator in fluid communication with said supply area;
a plurality of bags in fluid communication with said cellular growth area, said bags being adapted to provide fluids to said cellular growth area;
a fluid recirculation path in fluid communication with said cellular growth area; and
an incubator for receiving said disposable apparatus said incubator having means for controlling flow of fluids within said bioreactor.

11. The cell expansion system of claim 10 further comprising at least two sheets of membrane between two adjacent plates, and wherein said supply area is between said two sheets of membrane.

12. The cell expansion system of claim 11 further comprising a support structure between said at least two sheets of membrane.

13. The cell expansion system of claim 12 wherein said support structure is a mesh.

14. The cell expansion system of claim 11 further comprising means for directing fluid into an area between adjacent sheets of membrane and means for directing another fluid into an area between a sheet of membrane and a plate.

15. The cell expansion system of claim 14 wherein the fluid between the membrane and a plate contains cells and the fluid between the two membranes supplies nutrients.

16. The cell expansion system of claim 10 wherein at least a surface of said plate facing a sheet of membrane has a corrugated surface.

17. The cell expansion system of claim 16 wherein at least a surface of said plate facing a sheet of membrane has a plurality of grooves, whereby the surface area of said plate is increased.

18. The cell expansion system of claim 17 wherein said grooves are parallel to each other.
